(19) United States
(12) Patent Application Publication
(10) Pub. No.: US 2021/0363687 A1
(43) Pub. Date: Nov. 25, 2021

(54) WASH CYCLE INDICATOR FOR TEXTILE GARMENTS

(71) Applicant: Textile-Based Delivery, Inc., Conover, NC (US)

(72) Inventors: Margaret Anne Monaco, Cary, NC (US); Ngoc Hong Tu Nguyen, Greensboro, NC (US); Jordan Schindler, Charlotte, NC (US); Michael A. Drzewinski, Lake Hopatcong, NJ (US); Nathan Jablonske, Hickory, NC (US)

(73) Assignee: Textile-Based Delivery, Inc., Conover, NC (US)

(21) Appl. No.: 17/326,799

(22) Filed: May 21, 2021

Related U.S. Application Data
(60) Provisional application No. 63/029,125, filed on May 22, 2020.

(51) Int. Cl. D06H 3/02; G01N 21/77; G01N 21/84; G01N 33/36; D06P 1/00

(52) U.S. Cl. CPC D06H 3/02; G01N 21/77; G01N 2021/8444; G01N 33/367; D06P 1/0096; G01N 21/84

(57) ABSTRACT

A textile wash cycle indicator system comprising a textile fiber or yarn or section comprising a dye system comprising at least one dye, the dye system configured to release an amount of the at least one dye during each consecutive wash cycle; wherein the release of the amount of the at least one dye correlates with the consecutive wash cycles, and a method of using same.

### CROSS-REFERENCE TO RELATED APPLICATIONS

This application claims the benefit of U.S. Provisional Application No. 63/029,125 filed on May 22, 2020, the entirety of which is incorporated herein by reference.

### TECHNICAL FIELD

The present disclosure provides an indicator that presents a visual change as a function of number of washes for a textile.

### BACKGROUND

Medicated yarns are woven or knitted into garments, like compression wear, socks, and leggings. The medicated yarns include, for example, an active ingredient configured to administer to the wearer during use for pain relief. The active ingredient content of in the garment decreases during use as well as after laundering, i.e., wash cycles. Current garments incorporating medicated yarns do not provide the consumer an indication of the number of wash cycles.

### SUMMARY

In one example, a textile wash cycle indicator system is provided, the indicator system comprising an initial amount of a dye system that comprises at least one dye, the dye system configured to release an amount of the at least one dye during consecutive wash cycles. The system also includes where the release of the amount of the at least one dye correlates with the consecutive wash cycles.

In another example, the release of the at least one dye correlates with a visual change of the dye system. In another example, alone or in combination with any of the previous examples, the visible change is: a change of color; producing, reducing, or increasing a visual presence of a symbol; producing, reducing, or increasing a visual presence of text characters; or combinations thereof. In another example, alone or in combination with any of the previous examples, the visible change is irreversible.

In another example, alone or in combination with any of the previous examples, the dye system is integral with a textile. In another example, alone or in combination with any of the previous examples, the dye system is integral with a yarn or fiber of a textile.

In another example, alone or in combination with any of the previous examples, the dye system may include: a first dye having a first washing fastness value; and one or more dyes different from the first dye, each of the one or more dyes different from the first dye independently having a washing fastness value less than the first wash fastness value. In another example, alone or in combination with any of the previous examples, the rate of change of at least one of the washing fastness values less than the first wash fastness value corresponds to a first range of wash cycles.

In another example, alone or in combination with any of the previous examples, the rate of change of at least one of the washing fastness values less than the first wash fastness value corresponds to a second range of wash cycles greater than or less than the first range of wash cycles. In another example, alone or in combination with any of the previous examples, the rate of change of at least one of the washing fastness values less than the first wash fastness value corresponds to a range of wash cycles between 1 and about 10 and where the rate of change of at least one of the washing fastness values less than the first wash fastness value corresponds to a range of wash cycles of more than about 10.

In another example, alone or in combination with any of the previous examples, the rate of change of at least one of the washing fastness values less than the first wash fastness value corresponds to a range of wash cycles between 1 and about 10 and where the rate of change of at least one of the washing fastness values less than the first wash fastness value corresponds to a range of wash cycles of between about 10 to about 20 and where the rate of change of at least one of the washing fastness values less than the first wash fastness value corresponds to a range of wash cycles greater than 20.

In another example, alone or in combination with any of the previous examples, the first dye or the one or more dyes different from the first dye are coupled to or immobilized on an inorganic support or an organic support. In another example, alone or in combination with any of the previous examples, the first dye and the one or more dyes different from the first dye are coupled to or immobilized on an inorganic support or an organic support.

Another example includes a method for indicating wash cycles of a textile. The method also includes providing an indicator associated with a yarn, fiber, or section of fabric of the textile that directly or indirectly corresponds to a qualitative, semi-qualitative, quantitative or semi-quantitate amount of wash cycles of a textile. The method also includes providing a visual change of the indicator that directly or indirectly corresponds to the qualitative or the quantitate amount of washing cycles of the textile.

### BRIEF DESCRIPTION OF THE DRAWINGS

FIG. 1 depicts a garment with wash cycle indicator as disclosed and described herein.

FIG. 2 depicts an exemplary qualitative, semi-qualitative, quantitative or semi-quantitative wash cycle indicator as disclosed and described herein.

FIG. 3 depicts an exemplary qualitative, semi-qualitative, quantitative or semi-quantitative wash cycle correlation grid as disclosed and described herein.

FIG. 4 depicts regression of ΔE₀₀ verses wash cycles for an exemplary dye system with single dye indicator as disclosed and described herein.

FIG. 5 depicts regression of ΔE₀₀ verses wash cycles for an exemplary dye system with dual dye indicators as disclosed and described herein.

FIG. 6 depicts regression of ΔE₀₀ verses wash cycles for and exemplary dye system with multiple dye indicators as disclosed and described herein.

FIG. 7 depicts regression of ΔE₀₀ verses wash cycles for another exemplary dye system with multiple dye indicators as disclosed and described herein.

FIG. 8 depicts an exemplary dye system indicator production process as disclosed and described herein.

### DETAILED DESCRIPTION

Washing life-span is useful for fabrics and garments. In one example, washing lifespan is useful for fabrics and garments containing medicaments imbibed or incorporated in the yarn or fibers thereof. The present disclosure provides an indicator that will indicate qualitatively or quantitatively a predetermined number of wash cycles for the fabric or garment. In one example, the predetermined number of wash cycles is at least 1, 5, 10, 15, 20, 25 or more wash cycles. In one example, a perceivable indicia change can be observed after each subsequent wash throughout 5, 10, 15, or 25 or more wash cycles.

The present disclosure provides a wash cycle indicator suitable for a fabric or garment, the indicator configured to alter its visual appears in qualitative, semi-qualitative, quantitative, or semi-quantitative relationship to the number of wash cycles of the fabric or garment. In one example, the indicator is configured for reflecting approximately 15, approximately 20, and/or approximately 25 washes or more of the fabric of garment. In another example a single dye system is constructed for providing visual indication of approximately 15, approximately 20, and/or approximately 25 washes or more of the fabric of garment.

In one aspect of the present disclosure, the present system comprising of one or more dyes associated with a fiber, yarn or section of a fabric or garment is configured such that during or after each successive wash, a small amount of the one or more dye will fade out and after a predetermined number of washes, the dye will have completely faded out. In another aspect of the present disclosure, the present system comprising of one or more dyes associated with a fiber, yarn or section of a fabric or garment is configured such that during or after each successive wash, a small amount of the one or more dye will fade out and after 5 washes, 10 washes, 15 washes, 20 washes or 25 washes, etc., with the dye having completely faded out at its predetermined wash cycle limit. For example, the yarn/fabric or textile could start out with an initial visual indication and after a predetermined number of washes, would present a visual indication different from the initial visual indication.

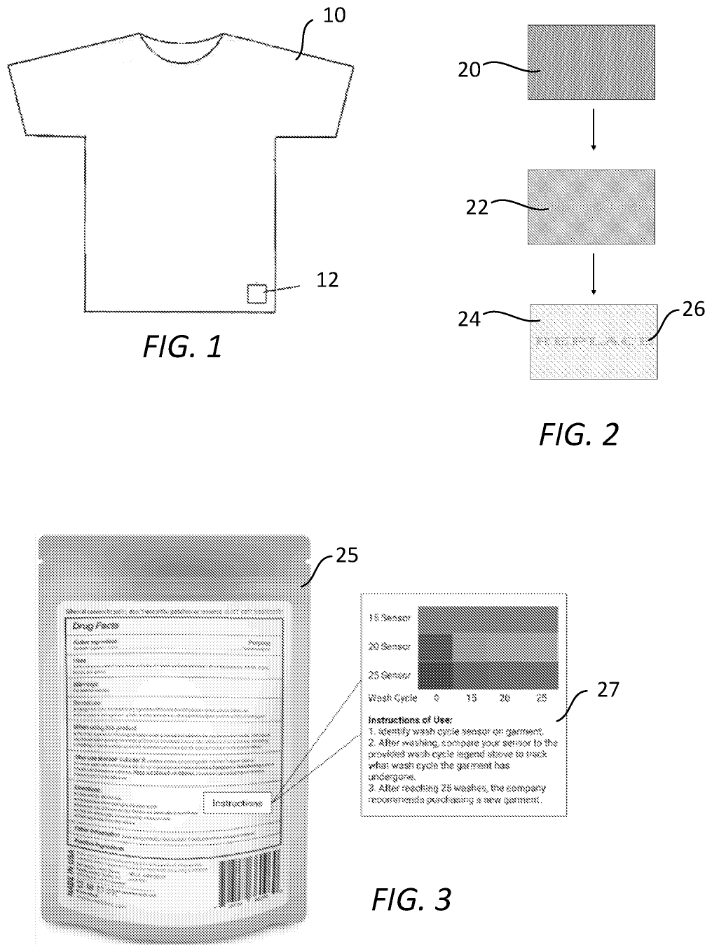

With reference to FIG. 1, and exemplary garment 10 is shown having exemplary indicator 12, which may optionally include or be a part of a care label 13 or a separate section of garment 10. In one example, indicator 12 is comprised of the same or different fabric of the garment. Exemplary indicator 12 is shown in FIG. 2 which undergoes a visual transition corresponding to one or more wash cycles of the indicator. In one example, indicator 12 has an initial visual indication 20 which may represent the end users purchased product prior to the end user performing a wash cycle on the product. After one or more washes, exemplary indicator 12 transitions to an intermediate visual indication 22. After reaching a predetermined number of wash cycles, the exemplary indicator 12 transitions to a substantially final visual indication 24. In one example, the intermediate or final visual indication presents text 26 or one or more symbols. The text 26 or symbols can be provided by screen printing the dye(s) or other methods. In an alternate embodiment, the indicator 12 undergoes a continuous, semi-continuous or stepwise visual transition and the visual transition is referenced on a grid 27 for providing a frame of reference to determine qualitatively or quantitatively the number one cycles experience by the product. Such grid 27 can be provided on the product packaging or available on the manufacturer's website, for example.

### Dyes of Dye System

In one example, the dye system comprises at least one dye chosen from semi-permanent hair dyes. Semi-permanent hair dyes typically are configured for release from hair over the course of consecutive shampoos. The dye system of the present disclosure can include one or more dyes selected from the five main classes of dye: acid dyes, direct dyes, disperse dyes, mordant dyes, and basic dyes. In another example, the dye system of the present disclosure can include one or more dyes having chemical structures that were of medium molecular weight (less than 1000 Mw) and contained at least one acid dye.

In another example, the dye system of the present disclosure includes one or more acid dyes comprising a sulfonate auxochrome group that are negatively charged (anionic) compounds which denature or lose color fastness under alkaline pH.

In yet another example, the dye system of the present disclosure includes one or more basic dyes that are cationic (positively charged) compounds. In another example, the dye system of the present disclosure includes one or more direct dyes that are cationic or anionic, highly soluble in water and applied to the fibers/yarns in solutions containing ionic salts and electrolytes. In yet another example, the dye system of the present disclosure includes one or more disperse dyes that are used for dyeing synthetic fibers such as cellulose acetate, polyester, and nylon. In yet another example, the dye system of the present disclosure includes one or more mordant dyes that use mordant for improving the color retention property of the dye molecules within the fiber.

In one example, the dye system comprises at least one dye providing a linear rate of change of a visual indicia (e.g., wash fastness) with respect to successive wash cycles. In another example, the dye system comprises at least one dye providing a polynomial rate of change with respect to successive wash cycles. In another example, the dye system comprises at least one dye providing a linear rate of change of a visual indicia with respect to successive wash cycles in combination with one or more dyes providing a polynomial rate of change with respect to successive wash cycles.

### Exemplary Dye Systems

In one example, the dye system is based on a visual indicia, based on or correlated to, a ΔE equation. There are a number of ΔE equations that can be used to calculate the difference between two colors or a change of color. In one example, the equation for ΔE₂₀₀₀ (=ΔE₀₀)was used. The numerical value for ΔE₀₀ can be correlated to a human's eye perception of color change approximately 75-80% of the time. Alternatively, the ΔE_{Lab} equation can be used.

Using ΔE values to correlate rate of color change over the course of 25 washes can provide satisfactory results for dye systems that present linear or polynomial wash fastness. In one example, a change every 5 wash cycles of about 5 points is used such that the color change is perceivable to the human eye for dye systems that present linear wash fastness. If the wash fastness curve is polynomial, the curve can plateau at a certain wash cycle value with no further change but there will still be a difference to the end-user leading up to the point of plateau. In one example, the presently disclosed dye systems provide linear rates of change in ΔE versus number of wash cycles, polynomial rates of change in ΔE versus number of wash cycles, or or combinations thereof. The dye systems of the present disclosure provide qualitative or quantitative wash cycle indicators providing end-user indication of one or more, for example 5, 15, 20, and 25 wash cycles.

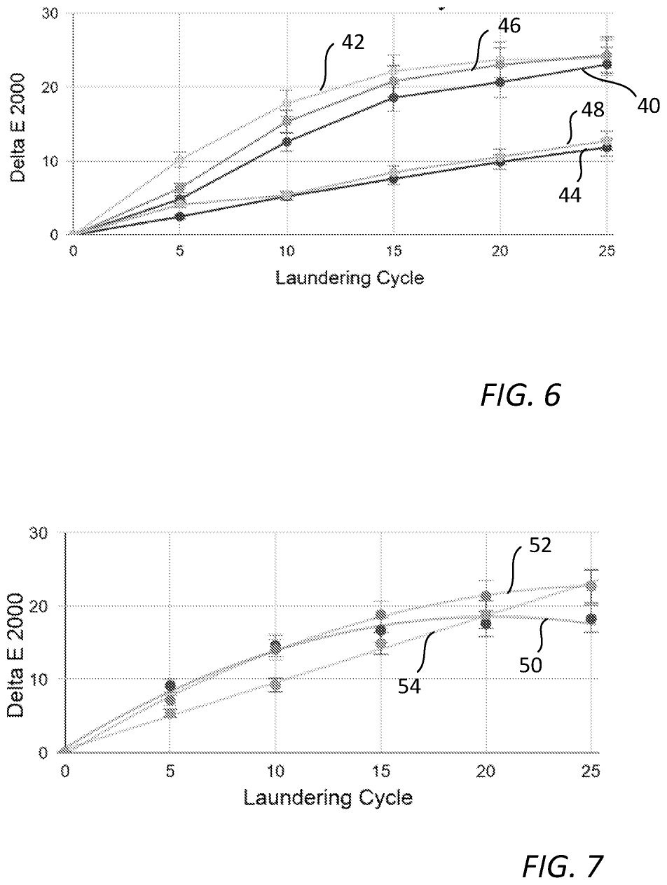

In one example, a single dye was used in the dye system with dye add-on %, prepared as a dye system indicator for use with fabric or textiles. Add-on % was done by determining the weight of the un-dyed fabric and taking 1% of that weight to as the add-on amount needed. For example, fabric samples were about 10 grams, so to get the amount of dye added to the bath, 1% of 10 grams 0.10 grams of dye was added (1% of 10 grams). Dyes were not added together in the same bath, rather the fabric was dyed in two different steps using separate dye baths. Thus, for example combinations that were 1% dye A and 1% dye B, with the total add-on % being 2%, was achieved with separate dye baths at a concentration that was 1% of each dye. In one example, a plurality of different percent add-ons for a single dye, were used for a predetermined number of wash cycles. Thus, as shown in FIG. 5, Basic Blue 3 with 0.5% Basic Blue 3 add-on (30); 1% Basic Blue 3 add-on (32); and 2% Basic Blue 3 (34) was used for a predetermined wash cycle indicator, i.e., 20 wash cycles. As shown in FIG. 6, the regression of ΔE₀₀ values vs. wash cycles for the different percent add-ons for Basic Blue 3, determined at 5 wash cycle individuals up to a total of 20 wash cycles is shown, and provides a functioning predetermined wash cycle indicator for fabric or textiles.

In one example, combinations of one or more dyes are used, where one dye has good wash fastness (permanent dye, ΔE₀₀<10) and another dye has bad wash fastness (leaving dye, AE₀₀>20), using only 1% add-ons of each dye.

In one example, a dual dye, two color dye system was used. Determination of color combinations was determined based on whether or not the dyes were permanent (high color fastness/ ΔE <10) or transient (low color fastness/ΔE>20) and by ensuring that color combinations would result in visual changes, for example, such as blue/yellow/green, and red/blue/purple combinations. As shown in FIG. 6, a combination of blue and yellow dyes, or blue and red or purple dyes, are used and ΔE₂₀₀₀ vs. wash cycle was determined. The dual dye, 2-color dye systems were Basic Blue 3/Acid Yellow 17 with formic acid (42); Blue 3/Direct Yellow 12 (40); Basic Blue 3/Mordant Yellow 3 (46); Acid Blue 45/Basic Violet 10 (44); and Disperse Red 167/Direct Blue 86 (48). The data shows that the 2-color dye systems of Basic Blue 3/Acid Yellow 17 with formic acid (32); Blue 3/Direct Yellow 12 (30); and Basic Blue 3/Mordant Yellow 3 (36); provided ΔE₂₀₀₀ values greater than 20 over 25 wash cycles, beginning at 15 wash cycles and thus provided wash cycle indicators for fabrics and textiles.

In one example, a 3 component dye system was used. As shown in FIG. 7, an exemplary dye system is shown comprising of three different combination of one or more indicator dyes, each combination configured to provide visual indication for a predetermined number of washes. For example, an exemplary 3-dye system comprised 0.5% Direct Blue 86 and 0.2% Basic Yellow 2 (50); 0.5% Basic Blue 3 (52); and 1% Basic Blue 3 and 2% Direct Yellow 12 (54), where the group of Direct Blue 86 and 0.2% Basic Yellow 2 was used for indicating a visual change after approximately 15 washes, where 0.5% Basic Blue 3 alone was used for indicating a visual change of approximately 20 washes; and the group of 1% Basic Blue 3 and 2% Direct Yellow 12 was used for indicating a visual change of approximately 25 washes. Thus, collectively, the 3 component dye system provided a visual indication suitable for use on a fabric or garment to indicate a wash cycle history of approximately 15 washes, proximally 20 washes, and approximately 25 washes.

The 1% Basic Blue 3 and 2% Direct Yellow 12 (used for indicating approximately 25 washes) and the 0.5% Basic Blue 3 (used for indicating approximately 20 washes) had a ΔE₀₀ values of 22.67 and 22.77, respectively. Comparison, 0.5% Direct Blue 86 and 0.2% Basic Yellow 2 after 15 washes had a ΔE₀₀ of 18.28. In one example, the one or more dyes of the dye system configured for providing visual indication of the maximum number of wash cycles, for example 25 or more washes, had a substantially linear regression of ΔE₂₀₀₀ value vs. wash cycles.

### Methods of Indicator Production

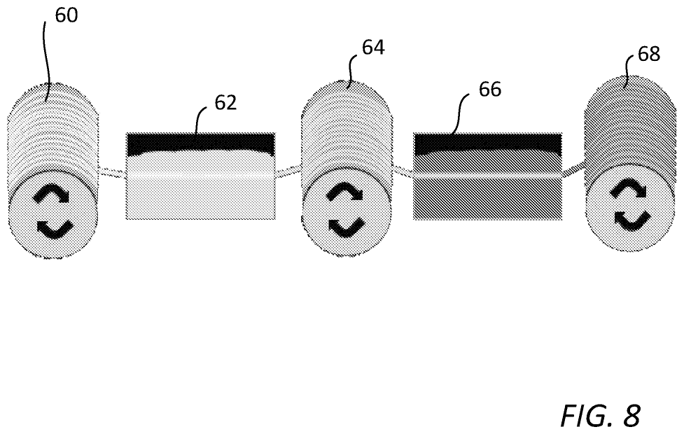

In one example, a continuous, semi-continuous, or batch yarn dyeing process can be implemented. In one example, a continuous dyeing process, as shown in FIG. 8, where feed roll 60 of yarn is introduced to first dye bath 62 and collected by take-up roll 64 and subsequently introduced to second dye bath 66 and collected on spool 68. In one example, dwell time in each of the dye baths is 30 minutes at a temperature of 100° C. Not depicted in FIG. 8 is the intermediary rinse and drying phase of the dyed yarns going from one dye bath to the other. A similar set-up can be used to continuously dye fabric through 3 or more dye baths.

In one example, a known initial dye bath concentration can be monitored using conventional techniques for a given period of time, and a dynamic concentration of the dye calculated which can be adjusted automatically to ensure that the same percent add-on is present in the dye bath. In one example, color differences can be determined using the yarn rather than the fabric and correlated to the visual change analysis for the fabric.

Thus, in one example a dye system comprising various two color combinations were washed and data was collected over the 25 wash cycles providing a functional wash cycle sensor for 15, 20, and 25 wash cycles. In another example, a percent add-on of dyes was determined to determine the rate of color change over the course of 25 washed and a best fit analysis was used to provide a 15, 20, and 25 wash cycle sensor. Thus, for example, an exemplary 2 color combination dye system was developed comprising 0.5% Direct Blue 86 and 0.2% Basic Yellow 2 for providing a visual indication of change after a 15 wash cycle, 0.5% Basic Blue 3 for providing a visual indication of change after a 20 wash cycle, and 1% Basic Blue 3 and 2% Direct Yellow 12 for providing a visual indication of change after a 25 wash cycle suitable for use for a fabric or garment.

The present disclosure provides for a continuous dye process that maximizes dye retention/wash fastness and maximizes indicator effectiveness. The present disclosure provides for a dye system compatible with various detergents, wash cycles, and anti-soiling performance of the indicator under various lighting settings.

The present dye system and indicator provides wide process latitude for accommodating wash cycles that can differ in the amount of water used, temperature, and time with acceptable dye release profiles throughout the lifetime of the garment while minimizing leaving dye on the skin during wear. The present dye system and indicator can be incorporated into textile products and/or constructed of natural, synthetic or blends of yarns, with substantially little if any compromise of the product's functionality and comfort. The present dye system and indicator is designed and configured for complimentary reading/indexing instructions that can be presented on product packaging.

### Experimental

The conditions of dye bath such as temperature and pH may be varied depending on the uniformity of the fabric and the degree of penetration of dyes in the fibers. The evaluation of dye systems suitable as indicators consisted of a CIE color difference test after washing in the standard washing machine and laundrometer. The visual change of the of the indicator was determined using AATCC EP7-EP7-2015: “Instrumental Assessment of the Change in Color of a Test Specimen.” In this procedure, a spectrophotometer and visual gray scale assessments were performed before and after treatment (wash cycle) to the fabric. A ΔE value is obtained for each specimen before and after each subsequent wash cycle. ΔE is calculated by using Equation (I):

ΔE=(ΔL²+Δa²+Δb²)/2 Equation (I)

where ΔL is the difference in lightness from black to white, Aa is the difference in color from red to green, and Δb is the difference in color from blue to yellow.

A threshold ΔE₀₀ value of about 20-50 was used so as to provide a range of acceptable values that would correlate to a detectable visual difference to the human eye. Values below 20 will likely have an unperceivable difference to the human eye or not be different in the case where the ΔE₀₀ value is initially 0. The ΔE₀₀ values were used to analyze the rates of color depth change for different percent add-on per fabric weight samples.

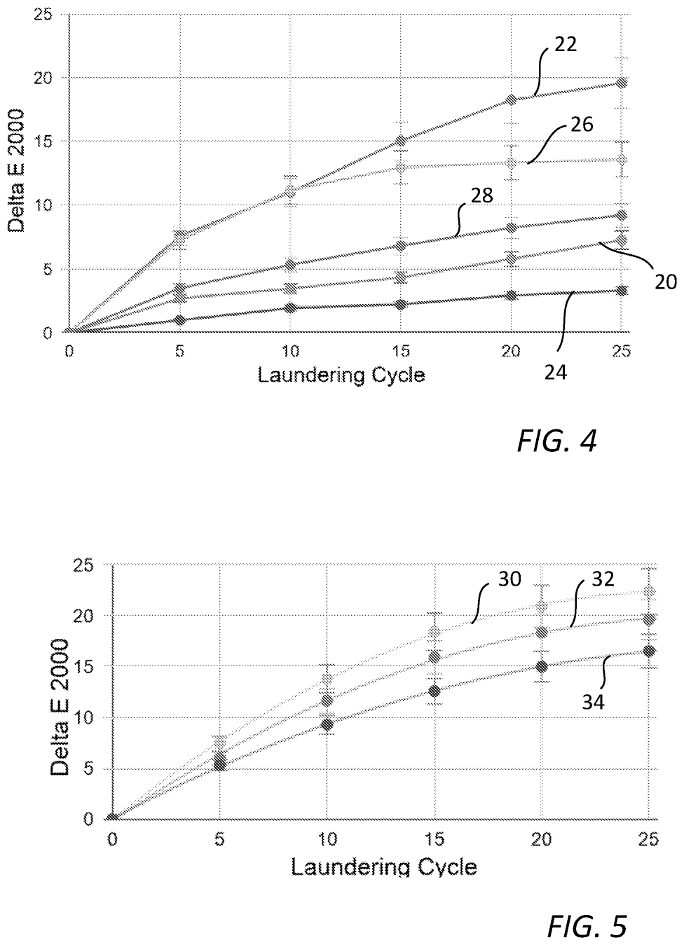

A stock solution using water and dye. was used to create dye baths in Ahiba-Labomat beakers with a maximum volume of 200 milliliters, in which samples were introduced. The Ahiba-Labomat was set to have the maximum ramp rate, to have a dwell time of 30 minutes, and to maintain a temperature of 100° C. After dyeing, the samples were rinsed and dried by first running the fabric through a padder and then laying the fabrics out to dry overnight in the open air. After drying, the samples underwent color assessment using aColor i7 Benchtop Spectrophotometer with the following settings: specular included, UV excluded, observer angle of 10°, illuminate D65, an average of 4 readings per sample, and medium to large aperture to obtain initial ΔE₀₀ values. All of the fabric samples were dyed using the same conditions and at a temperature of 100° C. for 30 minutes. After dyeing, the samples were washed 25 times to evaluate the degradation/color fastness of each exemplary dye, an example ΔE₀₀ verses wash cycles is shown in FIG. 4 for Basic Violet 10 (20) Basic Blue 3 (22) Basic Violet 3 (24) Basic Yellow 2 (26) Basic Red 18 (28).

Once the fabric samples were dyed, dried, and the initial ΔE₀₀ values obtained, a detergent solution of 0.37% AATCC detergent and warm water was prepared. One of the fabric samples, 200 milliliters of detergent solution, and 10 metal ball bearings were added to the launderometer beakers, which were subsequently sealed and then placed in the launderometer to run for 45 minutes at a temperature of 40° C. After a cycle of the launderometer finished, the fabric was, rinsed, padded, and placed in a drying oven for 20 minutes before measuring color change. Each fabric sample underwent 5 launderometer cycles which is equivalent to 25 traditional washing cycles and color was assessed after each cycle resulting in 6 different color readings, including the initial color assessment before laundering.

Wash cycle testing was performed in accordance with AATCC 61: “Test for Colorfastness to Laundering—Accelerated procedure.” For the washing procedure, the recommended washing instructions commencement for fabrics and garments containing medicament were used as wash cycle parameters, e.g., 0.37% AATCC laundering detergent, 10 ball bearings, temperature of 40° C., and a wash cycle of 45 minutes in a launderometer.

Although the above disclosure has been presented in the context of exemplary embodiments, it is to be understood that modifications and variations may be utilized without departing from the spirit and scope of the invention, as those skilled in the art will readily understand. Such modifications and variations are considered to be within the purview and scope of the appended claims and their equivalents.

We claim:

1. A textile wash cycle indicator system comprising:
a textile fiber, yarn, or section comprising an initial amount of a dye system comprising at least one dye, the dye system configured to change an amount of the at least one dye during consecutive wash cycles resulting in a visual change;
wherein the change of the amount of the at least one dye correlates with the consecutive wash cycles.

2. The textile wash cycle indicator system of claim 1, wherein the change of the amount of the at least one dye correlates with a visual change of the dye system.

3. The textile wash cycle indicator system of claim 1, wherein the visual change in the textile fiber or yarn or section is a change of color.

4. The textile wash cycle indicator system of claim 1, wherein the visible change is: a change of a color; producing, reducing, or increasing a visual presence of a symbol; producing, reducing, or increasing a visual presence of text characters; or combinations thereof.

5. The textile wash cycle indicator system of claim 1, wherein the visible change is irreversible.

6. The textile wash cycle indicator system of claim 1, wherein the dye system is a section selected from a care label, size label, or tag.

7. The textile wash cycle indicator system of claim 1, wherein the dye system is integral with a textile.

8. The textile wash cycle indicator system of claim 1, wherein the dye system is integral with a yarn or fiber of a textile.

9. The textile wash cycle indicator system of claim 1, wherein the dye system comprises:
a first dye having a first washing fastness value; and
one or more dyes different from the first dye, each of the one or more dyes different from the first dye independently having a washing fastness value less than the first wash fastness value.

10. The textile wash cycle indicator system according to claim 9, wherein the rate of change of at least one of the washing fastness values less than the first wash fastness value corresponds to a first range of wash cycles.

11. The textile wash cycle indicator system of claim 9, wherein the rate of change of at least one of the washing fastness values less than the first wash fastness value corresponds to a second range of wash cycles greater than or less than the first range of wash cycles.

12. The textile wash cycle indicator system of claim 9, wherein the rate of change of at least one of the washing fastness values less than the first wash fastness value corresponds to a range of wash cycles between 1 and about 10 and wherein the rate of change of at least one of the washing fastness values less than the first wash fastness value corresponds to a range of wash cycles of more than about 10.

13. The textile wash cycle indicator system of claim 9, wherein the rate of change of at least one of the washing fastness values less than the first wash fastness value corresponds to a range of wash cycles between 1 and about 10 and wherein the rate of change of at least one of the washing fastness values less than the first wash fastness value corresponds to a range of wash cycles of between about 10 to about 20 and wherein the rate of change of at least one of the washing fastness values less than the first wash fastness value corresponds to a range of wash cycles greater than 20.

14. A method for indicating wash cycles of a textile, the method comprising:
providing an indicator associated with a yarn, fiber, or section of fabric of the textile that directly or indirectly corresponds to a qualitative, semi-qualitative, quantitative or semi-quantitate amount of wash cycles of a textile;
providing a visual change of the indicator that directly or indirectly corresponds to the qualitative or the quantitate amount of washing cycles of the textile.

15. The method according to claim 14, wherein the visible change is: producing, reducing, or increasing a visual presence of a color; producing, reducing, or increasing a visual presence of a symbol; producing, reducing, or increasing a visual presence of text characters; or combinations thereof.

16. The method according to claim 14, wherein the indicator comprises a dye system, the dye system comprises:
a first dye having a first washing fastness value; and
one or more dyes different from the first dye, each of the one or more dyes different from the first dye independently having a washing fastness value less than the first wash fastness value.

17. The method according to claim 16, wherein the rate of change of at least one of the washing fastness values less than the first wash fastness value corresponds to a first range of wash cycles.

18. The method according to claim 16, wherein the rate of change of at least one of the washing fastness values less than the first wash fastness value corresponds to a second range of wash cycles greater than or less than the first range of wash cycles.

19. The method according to claim 16, wherein the rate of change of at least one of the washing fastness values less than the first wash fastness value corresponds to a range of wash cycles between 1 and about 10 and wherein the rate of change of at least one of the washing fastness values less than the first wash fastness value corresponds to a range of wash cycles of more than about 10.

20. The method according to claim 16, wherein the rate of change of at least one of the washing fastness values less than the first wash fastness value corresponds to a range of wash cycles between 1 and about 10 and wherein the rate of change of at least one of the washing fastness values less than the first wash fastness value corresponds to a range of wash cycles of between about 10 to about 20 and wherein the rate of change of at least one of the washing fastness values less than the first wash fastness value corresponds to a range of wash cycles greater than 20.
